(19) United States
(12) Patent Application Publication
(10) Pub. No.: US 2009/0076444 A1
(43) Pub. Date: Mar. 19, 2009

(54) METHOD AND APPARATUS FOR SECURING A NEUROMODULATION LEAD TO NERVOUS TISSUE OR TISSUE SURROUNDING THE NERVOUS SYSTEM

(75) Inventors: Andre G. G. Machado, Beachwood, OH (US); Ali R. Rezai, Shaker Heights, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/177,698

(22) Filed: Jul. 22, 2008

Related U.S. Application Data
(60) Provisional application No. 60/962,247, filed on Jul. 27, 2007.

(51) Int. Cl. A61M 37/00

(52) U.S. Cl. 604/82

(57) ABSTRACT

An apparatus for securing a neuromodulation lead to nervous tissue or surrounding tissue (i.e. meninges) is provided. The neuromodulation lead includes an elongated member having a proximal end, a distal end, and a passage extending there between. The lead further includes at least one electrical contact secured around an outer surface of the distal end of the elongated member for stimulating nervous tissue. The apparatus comprises first and second tubular members are disposed within the passage of the elongated member. Each of the tubular members terminates at the distal end of the elongated member. First and second glue components are provided for injecting into the first and second tubular members, respectively. The first and second glue components, when injected, exit the respective tubular members at the distal end of the elongated member and mix to create a solidified glue that anchors the distal end of the elongated member within the nervous tissue.

### RELATED APPLICATION

This application claims the benefit of U.S. Provisional Patent Application Ser. No. 60/962,247, filed Jul. 27, 2007 which is incorporated herein by reference.

### TECHNICAL FIELD

The present invention is directed to the field of neuromodulation and, in particular, is directed to a method and apparatus for securing the distal end of a neuromodulation lead to nervous tissue or tissue surrounding the nervous tissue (i.e. meninges).

### BACKGROUND OF THE INVENTION

Neuromodulation is one of the most important therapeutic methods of modern medicine. Neuromodulation has revolutionized the treatment of several neurological disorders, such as movement epilepsy, depression, obsessive-compulsive disorder and motor deficits are enormous. While the theoretical applications grow, technical and mechanical limitations create significant barriers to the final application of such methods in humans. One limitation is related to the positioning of the electrodes (also called electrode leads or leads) to the desired neural target. Precision is very important since it is desirable to selectively modulate only the neural circuits involved in a given disease. The rest of the circuitry must not be engaged in order to avoid side effects. As hard as it is to find the ideal location for a lead, it is often more difficult to keep the lead in position. This issue is particularly critical in situations where the lead is placed in natural spaces, in and around the neural structures. Such spaces are often larger than the dimensions of the leads, making migration an all too common occurrence due to the lack of support or adherence to the structures. It is usually the surgeon's choice to tack the lead in place with sutures or other methods, holding it in place and preventing migration. However, tacking is not possible when the leads are inserted percutaneously since the surgeon can only control the proximal end of the lead that is coming out of the entry site while the distal lead of the tip is “free” inside the body. Several structures can be reached percutaneously, such as the surface of the spinal cord, the epidural space, peripheral nerves, and the convexities of the brain. Spinal cord stimulation leads are more commonly used to treat chronic pain and became the prototypical situation in which lead migration is a serious issue, threatening the efficacy of neuromodulation.

Spinal cord stimulation is one of the treatment options considered for patients with chronic pain. Initially, the rationale for its application was based on the gate control theory. It was expected that stimulation of large diameter fibers in the spinal cord would close the gate for pain-carrying fibers of smaller diameters and thus modulate incoming pain stimuli. As experience built, it became evident that spinal cord stimulation was more effective in some cases than in others. Patients with appendicular neuropathic pain syndromes seem to compose a group in which spinal cord stimulation can be valuable as a treatment resource. In particular, failed-back surgery syndrome became the major indication for this kind of procedure, although other syndromes such as complex regional pain syndrome and angina pectoris can also benefit. The success rates for the procedure tend to vary from 55% to 70% and, while restricting patient selection is a key factor to increasing success rates, mechanical failure remains a major source of efficacy loss after implantation of the device. Lead migration accounts for a significant share in this failure group, being diagnosed in up to 27% of patients on postoperative follow-up.

One commonly used strategy is to implant percutaneous trial leads to be activated by an external pulse generator. The patients are then allowed to use the device for a few days in order to evaluate the degree of amelioration provided. Only patients with significant and consistent pain improvement have the permanent devices implanted.

The spinal cord stimulation (SCS) device consists of one or more leads, extension wires and an implantable pulse generator. The lead has, on one end, the active contacts that are placed over the dura mater in the spinal canal. It may have a single row, or two rows, of contacts which usually vary in number from four to eight per row. The material supporting the contact is most usually soft and flexible, thus preventing damage to the patient's tissues. The electrical wires that connect theses contacts are insulated in such a fashion that electricity is only delivered at the contacts. Another set of contacts, corresponding to the connector, is present at the other end of the lead. At this end, an extension wire is attached through a connection. This wire, in turn, is also connected to the implantable pulse generator so that the electrical current generated at this site will be ultimately delivered to the corresponding contacts on the active end of the lead.

It is often a difficult task to place SCS leads at the desired position in the spinal canal, just superficial to the dural sac. The exact position has to be found that provides coverage to the entire painful area in adequate intensities. After implantation, any movement (migration), even if minor, can cause changes in the stimulated area, leading to loss of coverage or unpleasant side effects. Side effects include stimulation of areas that are not included in the pain syndrome, sensations of burning and shock-like bolts, and inclusion of sensitive areas under the stimulation, such as the genitals. It is also possible to induce stimulation of the nerve roots instead of the spinal cord, causing painful stimuli to the abdomen, flank, and thorax. In short, lead displacement is a very undesirable event after implantation of spinal cord stimulators and mechanisms that can prevent its occurrence are desired.

Although migration can occur at any time after hardware implantation, it is more common in the first weeks after surgery. This is due to the scarring that is formed around an implantable device, including the leads inside the spinal canal. In re-operations, it is common to find a tunnel of scar tissue surrounding the lead, holding it in place. Therefore, developing strategies to prevent lead migration in the first weeks after implant are crucial in order to decrease the incidence of complications after these procedures. The space in which the SCS lead is implanted is almost virtual, since only a small amount of fat tissue separates the dural sac from the bone walls of the spinal canal. Hence, although migration in the anteroposterior direction is not possible, it can happen in the two other axis (lateral-lateral and inferior-superior). Inferior superior migration is at least partially prevented by anchoring the leads to the muscle fascia or to the skin, using anchoring devices already available in the industry. Since nothing holds the actual tip of the lead, preventing lateral-lateral (or medial-lateral) migration is very difficult, making migration in these directions more common and harder to deal with.

The present invention has been designed to prevent lead migration, particularly in the lateral-lateral and lateral-medial directions. The invention consists of a lead that holds, in addition to electrical wires, two built-in, parallel hollow tubes of small diameter. These tubes can carry two separate fluid components which are respectively applied to each tube. The components do not solidify until mixed together, which will only happen after delivery at the distal end of the lead. This glue will solidify at the distal tip of the lead, making it possible to prevent migration during the first and most crucial weeks, until scarring takes place.

### SUMMARY OF THE INVENTION

In accordance with the present invention, an apparatus for securing a neuromodulation lead to nervous tissue or tissue surrounding the nervous system (i.e. meninges) is provided. The neuromodulation lead includes an elongated member having a proximal end, a distal end, and a passage extending therebetween. The lead further includes at least one electrical contact secured around an outer surface of the distal end of the elongated member for stimulating nervous tissue. The apparatus comprises first and second tubular members are disposed within the passage of the elongated member. Each of the tubular members terminates at the distal end of the elongated member. First and second glue components are provided for injecting into the first and second tubular members, respectively. The first and second glue components, when injected, exit the respective tubular members at the distal end of the elongated member and mix to create a solidified glue that anchors the distal end of the elongated member within the nervous tissue.

In accordance with another exemplary embodiment of the present invention, a method is provided for securing a neuromodulation lead to nervous tissue or surrounding tissue. An elongated member having first and second tubular members disposed therein is provided. At least one electrical contact is secured around an outer surface of the elongated member. The at least one contact is placed into contact with the nervous tissue. First and second glue components are injected, respectively, into the first and second tubular members. The first and second glue components are extruded from the first and second tubular members, respectively, at a distal end of the elongated member. The first and second glue components mix and solidify to anchor the distal end of the elongated member to the nervous tissue.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIG. 1 is an illustration of an apparatus for stimulating nervous tissue;

FIG. 2 is an enlarged view of the proximal end of the lead of FIG. 1;

FIG. 3A is an enlarged view of the distal end of the lead of FIG. 1;

FIG. 3B is an enlarged view of the distal end of the lead in accordance with a second embodiment of the present invention;

FIG. 3C is an enlarged view of the distal end of the lead in accordance with a third embodiment of the present invention;

FIG. 4 is a section view of the lead of FIG. 1 taken along lines 4-4;

FIG. 5 is posterior view of the lead of FIG. 1 implanted within a spinal column;

FIG. 6 is a top view of the spinal column of FIG. 5;

FIG. 7 is a sectional view of the spinal column of FIG. 6 taken along lines 7-7;

FIG. 8 is a top view of the spinal column of FIG. 7;

FIG. 9 is an enlarged view of the distal end of the lead of FIG. 7; and

FIG. 10 is a sectional view of the lead of FIG. 9 taken along lines 10-10 following the application of glue.

### DETAILED DESCRIPTION

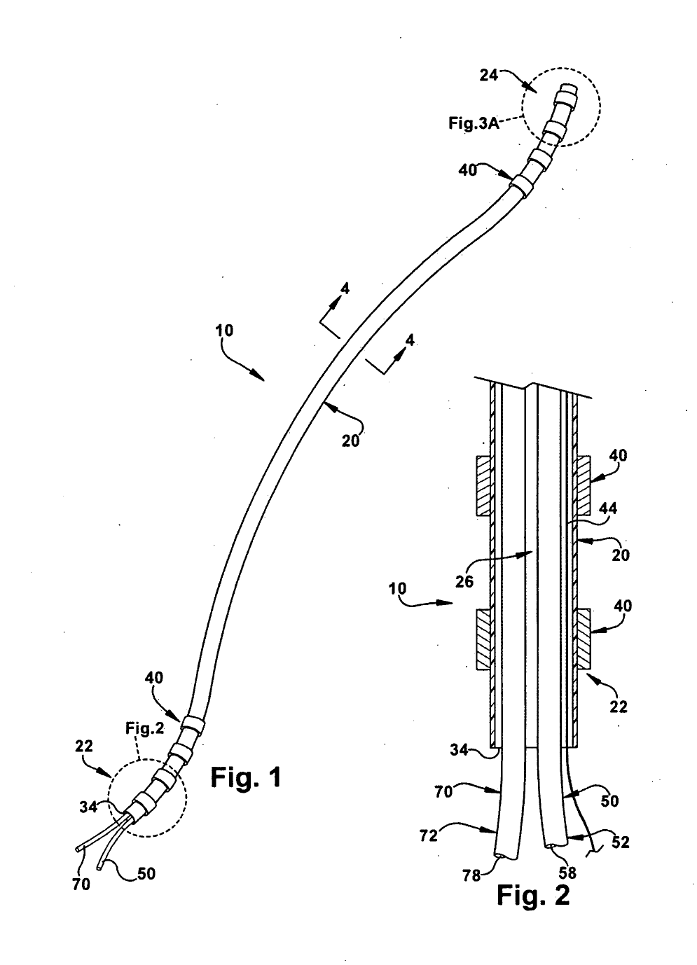

In accordance with the present invention, a neuromodulation lead 10 for stimulating nervous tissue is depicted in FIG. 1. The lead 10 comprises an elongated member 20 having a proximal end 22, a distal end 24, and a passage 26 extending therebetween. The passage 26 terminates at an opening 34 at the proximal end 22 and at the distal end 24 of the elongated member 20. The elongated member 20 is tubular in nature and preferably made of a soft, flexible material (such as any surgical grade tubing) that allows the elongated member 20 to deflect under axial force without plastic deformation to prevent damage to the patient's tissues during implantation. Although the elongated member 20 is depicted as having a substantially circular profile, those skilled in the art will appreciate that any other suitable profile may be used, such as square or rectangular.

One or more electrical contacts 40 are secured, or otherwise disposed, around the periphery of the elongated member 20 via glue or other suitable fastening means. The contacts 40 are generally circular in nature, and encircle a substantial portion, or all of, the periphery of the elongated member 20. Alternatively, the electrical contacts may vary in shape and designs using oval or square profiles are also possible. The electrical contacts 40 may be comprised of any biocompatible, electrically conductive material. Preferably, the contacts 40 are disposed around both the proximal end 22 and distal end 24 of the elongated member 20. As will be discussed, the electrical contacts 40 are used to stimulate nervous tissue within the body of a patient. Although FIG. 1 depicts four contacts 40 disposed around both the proximal end 22 and distal end 24 of the elongated member 20, any number of contacts may be provided, with preferably four to eight at each end. The number of contacts 40 will vary depending on the tissue stimulation desired. Regardless of the number of contacts 40 utilized, the contacts are generally evenly spaced along each end 22, 24 of the elongated member 20.

The contacts 40 at the distal end 24 of the elongated member 20 are ultimately placed over the dura matter 106 in the spinal canal 104 to perform neuromodulation. Thus, these contacts 40 act as the active contacts of the lead. An implantable pulse generator (IPG—not shown) serves as a power supply and pulse generator for the active contacts. Electrical wires 44 (FIGS. 2-4) electrically connect the pulse generator to the contacts 40 at the proximal end 22 of the elongated member 20, and subsequently to the contacts 40 at the distal end 24 of the elongated member 20. Alternatively, extension wires of varying lengths can be used to connect the spinal cord stimulation lead to the IPG. During the trial period (used to determine efficacy—see background session), a disposable extension wire is typically used. This extension connects the proximal end of the lead (which is concealed under the skin to prevent contamination) to an external pulse generator. After the trial period is completed, and if there is intention to make the implant permanent, the externalized extension wire is disposed and a new, sterile extension can be implanted to connect the lead to the IPG. In other instances, the lead itself is directly connected to the IPG. The contacts 40 at the proximal end 22 of the elongated member 2Q thus serve as passive contacts in that they merely provide a means of relaying power from the IPG to the contacts 40 at the distal end 24 of the elongated member 20.

As shown in FIGS. 2-4, a plurality of tubular members 50, 70 are disposed within the passage 26 of the elongated member 20, and generally extend from the proximal end 22 to the distal end 24 of the elongated member 20. Although only a first tube 50 and a second tube 70 are shown, more tubular members could be utilized. FIG. 2 depicts the proximal end 22 of the elongated member 20, wherein the first and second tubular members 50, 70 are shown extending out through the opening 34 in the elongated member 20. Following the implant and delivery of the adhesive liquid material through the tubular components, these extensions will be removed. This is necessary to allow for connection of the proximal end of the lead to the extension wire or IPG. The first tubular member 50 has a proximal end 52, a distal end 54, and a passage 56 extending therebetween. Likewise, the second tubular member 70 has a proximal end 72, a distal end 74, and a passage 76 extending therebetween. The passage of each tubular member 50, 70 terminates with an opening at the distal end and an opening at the proximal end, respectively.

Electrical wires 44 are shown which provides electrical communication between the contacts 40 at the distal end 24 of the elongated member 20 and the contacts 40 at the proximal end 24 of the elongated member 20. The electrical wires 44 also electrically couple the contacts 40 to each other. It will be appreciated by those skilled in the art that although the contacts 40, are depicted as being wired in series with one another so that all contacts 40 are energized simultaneously the contacts 40 may also be wired in parallel such that selective energizing of each contact 40 can occur. In either case, the wires 44 run along the passage 26 and are secured at several points to an inner wall 38 of the elongated member 20 (FIG. 4). The wires 44 are insulated so that electricity is only delivered at the contacts 40. The wires 44 exit the opening at the proximal end of the elongated member 20 and couple to the implantable pulse generator to provide electricity to the contacts 40.

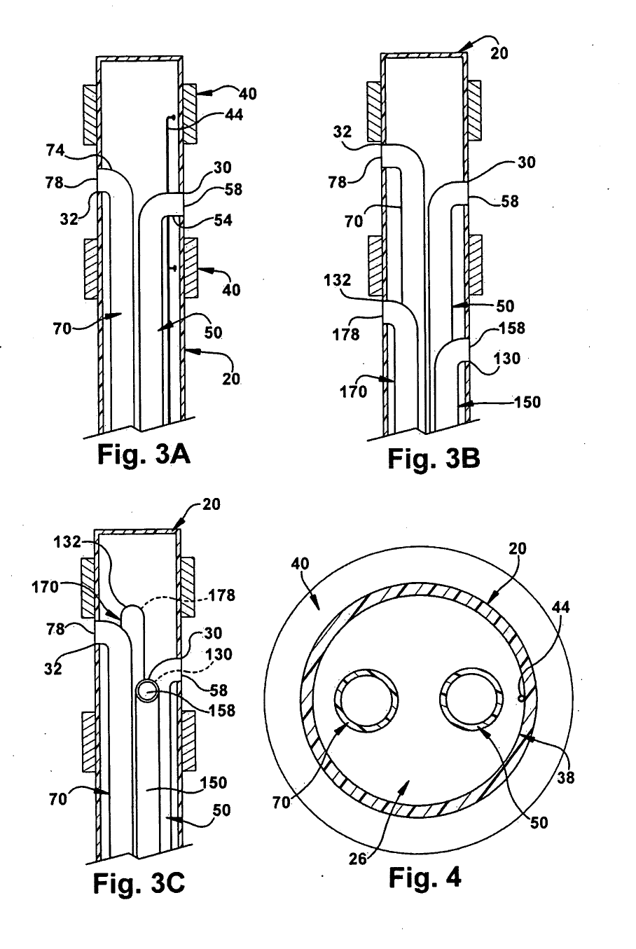

As shown in FIG. 3A, the distal end 24 of the elongated member 20 includes a plurality of apertures 30, 32 that extend from the outer surface 28 to the passage 26. The apertures 30, 32 are located around the periphery of the elongated member 20 and provide a means by which the distal end 54 of the first tubular member 50 and the distal end 74 of the second tubular member 70 are secured in place relative to the elongated member 20. The opening 58 at the distal end 54 of the first tubular member 50 is aligned with a first aperture 30 in the elongated member 20, and subsequently the distal end 54 of the first tubular member 50 is secured within the first aperture 30 with adhesive or the like. Likewise, the opening 78 at the distal end 74 of the second tubular member 70 is aligned with a second aperture 32 in the elongated member 20, and subsequently the distal end 74 of the second tubular member 70 is secured within the second aperture 32.

A third tubular member 150 and fourth tubular member 170 may also be provided, as shown in FIGS. 3B-3C. To accommodate the third tubular member 150 and fourth tubular member 170, the distal end 24 of the elongated member 20 includes third and fourth apertures 130, 132 that extend from the outer surface 28 to the passage 26. The third and fourth apertures 130, 132 are located around the periphery of the elongated member 20 and provide a means by which a distal end 154 of the third tubular member 150 and a distal end 174 of the fourth tubular member 170 are secured in place relative to the elongated member 20. An opening 158 at the distal end 154 of the third tubular member 150 is aligned with a the third aperture 130 in the elongated member 20, and subsequently the distal end 154 of the third tubular member 150 is secured within the third aperture 130 with adhesive or the like. Likewise, an opening 178 at the distal end 174 of the fourth tubular member 170 is aligned with the fourth aperture. 132 in the elongated member 20, and subsequently the distal end 174 of the fourth tubular member 170 is secured within the fourth aperture 132.

Multiple orientations of all the tubular members may be understood by those skilled in the art. For example, FIG. 3B depicts that the first tubular member 50 and second tubular member 70 are secured closest to the distal end 24 of the elongated member 20, while the third tubular member 150 and fourth tubular member 170 are secured proximal of the first tubular member 50 and second tubular member 70. Alternatively, the first tubular member 50, second tubular member 70, third tubular member 150, and fourth tubular member 170 may all be secured approximately the same distance from the distal end 24 of the elongated member 20, whereby each tubular member is secured in a different quadrant around the circumference of the elongated member 20. That is, the tubular members are spaced apart approximately 90 radial degrees from one another, as shown in FIG. 3C, although various spacing orientations around the periphery of the elongated member 20 are understood by those in the art.

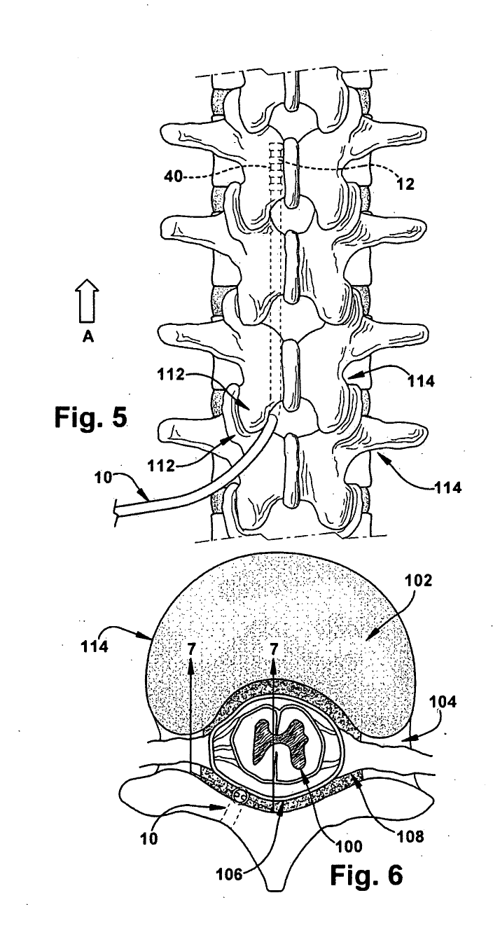

FIG. 5 depicts the lead 10 implanted into the spinal canal 104 of the patient. Once an incision has been made (not shown) in the patient's back, the distal end 12 of the lead 10 is inserted into the incision and, under fluoroscope guidance, the lead 10 is fed between the laminae 112 of adjacent vertebrae 114 to allow the lead 10 to access the epidural space 104 surrounding the spinal cord 100. Incising the skin is not necessary. Implantation is also possible through a needle that penetrates the skin, underlying tissues, muscle and spinal elements in a purely percutaneous fashion. The lead 10 is then fed in the direction A along the spine 100 towards the patient's head until the contacts 40 at the distal end 24 of the elongated member 20 are in proximity with the desired portion of the spinal cord 100 to be stimulated. The lead entries the spinal canal more frequently at the lumbar and thoracic levels. However, the distal tip of the lead will be more frequently implanted at the thoracic and cervical levels. Once the desired portion of the spinal cord 100 is reached the electrical contacts 40 are in the epidural space 106 (FIG. 6). Alternatively to this method of implantation, the lead can also enter the canal cranial to its desired final position and then be fed into the canal directed toward the feet in a “retrograde” fashion. The portion of the spinal cord 100 to be stimulated is selected based on the patient's particularized chronic pain or condition.

Although the illustrative figures depict use of the neuromodulation lead within the spinal canal 104 to perform SCS, those skilled in the art will recognize that alternative nervous tissue may be targeted within the patient, such as the cauda equine, nerve roots, Gasserian ganglion, cranial nerves, peripheral nerves such as the occipital nerves, and autonomic nervous system targets such as the sympathetic ganglia.

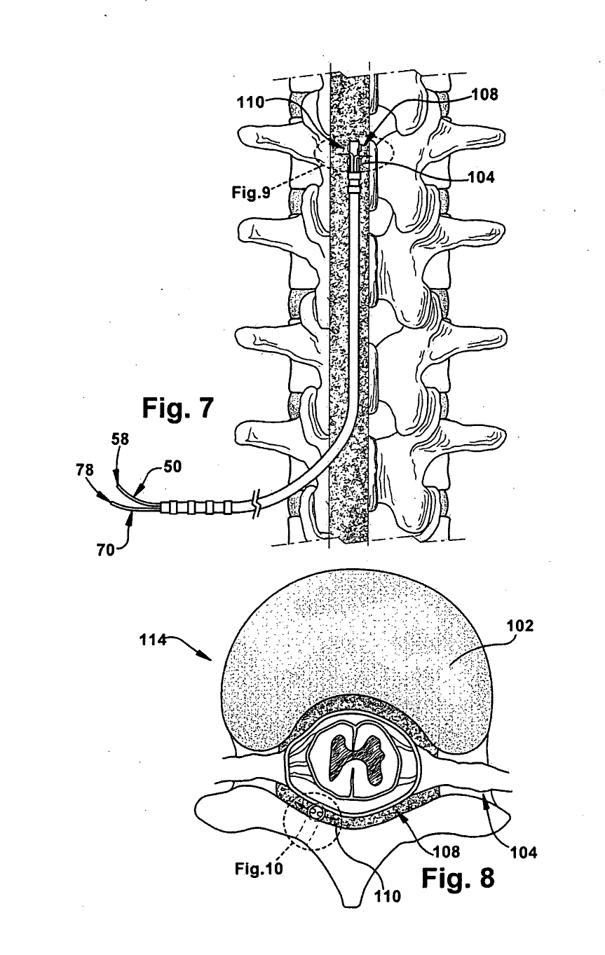

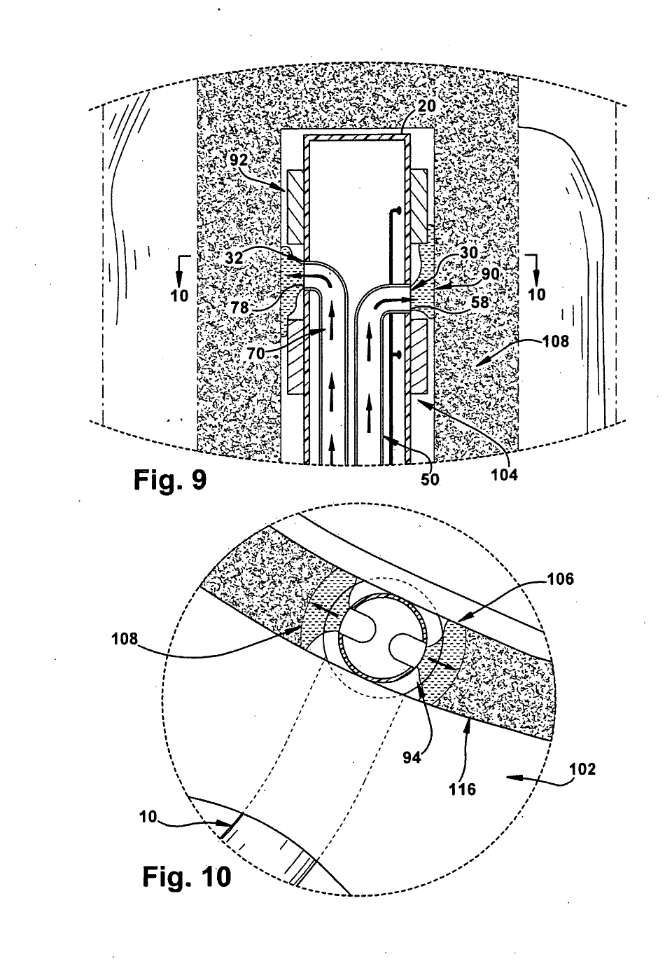

As shown in FIGS. 7-8, the positioning of the lead 10 within the epidural space 104 results in a void created in the fat 108 within the epidural space 104. Since this fat 108 is not rigid enough to maintain the position of the distal end 12 of the lead 10, glue components 90, 92 are used for stabilization. A first glue component 90 is injected into the proximal end 52 of the first tubular member 50, and a second glue component 92 is injected into the proximal end 72 of the second tubular member 70. Since the first and second tubular members 50, 70 are non-communicative, each glue component 90, 92 travels separately from the proximal end 22 of the elongated member 20 to the distal end 24 of the elongated member 20 as indicated by arrows B and C (FIG. 9).

Where the lead 10 includes a third and fourth tubular member 150, 170, the first glue component 90 is also injected into the proximal end of one of the third and fourth tubular members 150, 170, and the second glue component 92 is injected into the proximal end of the remaining tubular member. Therefore, as with the first and second tubular members 50, 70, each glue component 90, 92 travels separately through the third and fourth tubular members 150, 170 from the proximal end 22 of the elongated member 20 to the distal end 24 of the elongated member 20 (not shown).

Preferably, about 1-25 cc of fibrin glue is used to stabilize the distal end 12 of the lead 10. However, depending on the type of glue, more or less volume may be required. Therefore, the first and second glue components 90, 92 will be a human-animal fibrinogen/plasma solution and a thrombin solution, respectively. The fibrinogen may likewise be supplied from the blood of the patient. Additional components can be added to the solutions for preservation and packaging or for enhanced efficacy. Examples are fibrinolysis inhibitor products, calcium chloride, collagen or micro-collagen. Alternatively, any other biocompatible multi-part glue composite could be used. Where the glue composite utilized is comprised of three or more components, a corresponding number of tubular members and apertures must likewise be utilized to provide the same non-communicative flow path for each glue component until each glue component reaches the distal end 12 of the lead 10 to commence mixing. It is important to note that the glue is utilized to merely stabilize the distal end 12 of the lead 10, and is not meant to anchor the lead 10 indefinitely. Thus, the type of glue composite chosen must not be one which binds the distal end 12 of the lead 10 to the surrounding tissue forever. The glue is provided to temporarily stabilize the lead 10 until sufficient scar tissue builds. This is an important feature to allow for explantation (removal) of the implanted hardware, if need arises. A typical situation in which explantation is necessary is when an infection occurs.

As shown in FIG. 9, the first glue component 90 is extruded from the opening 58 in the first tubular member 50 at the first aperture 30 in the elongated member 20, and the second glue component 92 is extruded from the opening 78 in the second tubular member 70 at the second aperture 32 in the elongated member 20. This allows the first glue component 90 and the second glue component 92 to mix in the area surrounding the distal end 24 of the elongated member 20. If needed, rotation of the elongated member 20 by the surgeon facilitates this mixing process. The mixing of the first and second glue components 90, 92 results in a solidified glue 94 that surrounds the distal end 24 of the elongated member 20 (FIG. 10). This glue 94 anchors the distal end 24 of the elongated member 20, and thus the distal end 12 of the lead 10, between the dural sac 106 and a wall 116 of the vertebral body or laminae 112 of vertebrae 114, and keeps the distal end 24 of the elongated member 20 from moving in any direction, particularly the lateral-lateral, lateral-medial, and inferior-superior directions. Therefore, once the lead 10 has been orientated correctly relative to the desired portion of the spinal cord 100, the glue 94 ensures the surgeon that accurate neuromodulation stimulation is performed until sufficient scarring occurs in the epidural space 104 to naturally hold the distal end 12 of the lead 10 in place. The glue 94 is expected to be reabsorbed into the patient within a few weeks of implantation to allow for adequate scarring time.

Where third and fourth tubular members 150, 170 are used, the same mixing of the glue components 90, 92 and anchoring of the distal end 24 of the elongated member 20 occur. The lead 10 will be anchored along a longer portion of the elongated member 20 in the configuration of FIG. 3B, and a larger portion of the periphery of the elongated member 20 will be anchored in the configuration of FIG. 3C, as opposed to the two elongated member configuration of FIG. 3A. The nature of the patient's anatomy and procedure will dictate which configuration the surgeon decides to utilize in order to secure the lead 10 between the dural sac 106 and a wall 116 of the vertebral body.

Once the distal end 12 of the lead 10 has been anchored, the wires 44 are coupled to the pulse generator and the pulse generator is implanted within the patient (not shown). The pulse generator, having already been programmed accordingly, subsequently delivers electricity to the contacts 40 to perform the neurostimulation and treat the patient.

From the above description of the invention, those skilled in the art will perceive improvements, changes and modifications. Such improvements, changes and modifications within the skill of the art are intended to be covered by the appended claims.

Having described the invention, the following is claimed:

1. An apparatus for securing a neuromodulation lead to nervous tissue or surrounding tissue, the neuromodulation lead including an elongated member having a proximal end, a distal end, and a passage extending therebetween, the lead further including at least one electrical contact secured around an outer surface of the distal end of the elongated member for stimulating nervous tissue, said apparatus comprising:
first and second tubular members disposed within the passage of the elongated member, each of the tubular members terminating at the distal end of the elongated member; and
first and second glue components for injecting into the first and second tubular members, respectively,
the first and second glue components, when injected, exiting the respective tubular members at the distal end of the elongated member and mixing to create a solidified glue that anchors the distal end of the elongated member within the nervous tissue.

2. The apparatus of claim 1, wherein the first glue component is a human-animal fibrinogen plasma solution and the second glue component is a thrombin solution.

3. The apparatus of claim 2, wherein the plasma solution further comprises at least one component selected from the group consisting of fibrinolysis inhibitor products, calcium chloride, collagen, and micro-collagen.

4. The apparatus of claim 2, wherein the thrombin solution further comprises at least one component selected from the group consisting of fibrinolysis inhibitor products, calcium chloride, collagen, and micro-collagen.

5. The apparatus of claim 1, wherein the distal end of the elongated member includes first and second openings, each of the first and second openings extending from an outer surface of the elongated member to the passage.

6. The apparatus of claim 5, wherein the distal end of the first tubular member is secured to the first opening, and the distal end of the second tubular member is secured to the second opening.

7. The apparatus of claim 6, wherein the distal end of the elongated member further includes third and fourth openings, each of the third and fourth openings extending from the outer surface of the elongated member to the passage.

8. The apparatus of claim 7 further comprising third and fourth tubular members, wherein a distal end of the third tubular member is secured to the third opening, and a distal end of the fourth tubular member is secured to the fourth opening.

9. A method for securing a neuromodulation lead to nervous tissue or surrounding tissue, the method comprising the steps of:
providing an elongated member having first and second tubular members disposed therein, and at least one electrical contact secured around an outer surface of the elongated member;
placing the at least one contact in the vicinity of nervous tissue;
injecting first and second glue components, respectively, into the first and second tubular members; and
extruding the first and second glue components from the first and second tubular members, respectively, at a distal end of the elongated member, whereby the first and second glue components mix and solidify to anchor the distal end of the elongated member to the nervous tissue.
